(19) United States
(12) Patent Application Publication
(10) Pub. No.: US 2026/0134988 A1
(43) Pub. Date: May 14, 2026

(54) Patient Monitoring Method for Patient Waiting Areas

(71) Applicant: Mark Dickson, Orangeburg, SC (US)

(72) Inventor: Mark Dickson, Orangeburg, SC (US)

(21) Appl. No.: 18/944,042

(22) Filed: Nov. 12, 2024

(51) Int. Cl. G16H 40/67; G16H 40/20; G16H 50/30

(52) U.S. Cl. CPC G16H 40/67; G16H 40/20; G16H 50/30

(57) ABSTRACT

A method wear patients visiting a medical facility looking for treatment goes through an intake procedure checking vital patient functions, and then is issued a patient monitoring device that send messages to a processor that monitors each patient and alerts staff when vital patient functions change in an alarming way so the patient can go through the intake procedure again to determine if they need more expedited treatment.

### FIELD OF THE INVENTION

Class: A61B5/14551. Measuring characteristics of blood in vivo, e.g. gas concentration, pH value. Measuring characteristics of body fluids or tissues, e.g. interstitial fluid, cerebral tissue using optical sensors, e.g. spectral photometrical oximeters for measuring blood gases

### BACKGROUND

In the medical environment today, there are often long waits at a doctor's office, urgent cares or emergency rooms. After an initial intake examination people are categorized by how severe their medical situation is and then they are sent out in the waiting room until their time for seeing the doctor arrives. The problem with this process is that if a patient's status or illness changes, the staff at the medical service site has no idea the situation has deteriorated unless someone bring sit to the attention of the staff. The current situation does not give any real time information o the staff about patient changes.

This is unfortunate as patient monitoring devices are available. Wearable devices configured to monitor physiological parameters of a wearer can include: a physiological parameter measurement sensor which may configure to monitor a plurality of physiological parameters to communicate with a hardware processor with a display available to the medical office.

### SUMMARY—SENSORS

Sensors available today monitor a large number of patient vital signs. A practical application of this technique is pulse oximetry or plethysmography, which utilizes a noninvasive sensor to measure oxygen saturation and pulse rate, among other physiological parameters. Pulse oximetry or plethysmography relies on a sensor attached externally to the patient (typically for example, at the fingertip, foot, ear, forehead, or other measurement sites) to output signals indicative of various physiological parameters, such as a patient's blood constituents and/or analytes, including for example a percent value for arterial oxygen saturation, among other physiological parameters. The sensor has at least one emitter that transmits optical radiation of one or more wavelengths into a tissue site and at least one detector that responds to the intensity of the optical radiation (which can be reflected from or transmitted through the tissue site) after absorption by pulsatile arterial blood flowing within the tissue site. Based upon this response, a processor determines the relative concentrations of oxygenated hemoglobin (HbO₂) and deoxygenated hemoglobin (Hb) in the blood so as to derive oxygen saturation, which can provide early detection of potentially hazardous decreases in a patient's oxygen supply, and other physiological parameters.

Patient monitoring devices available today can include a plethysmograph sensor. The plethysmograph sensor can calculate oxygen saturation (SpO₂), pulse rate, a plethysmograph waveform, perfusion index (PI), pleth variability index (PVI), methemoglobin (MetHb), carboxyhemoglobin (CoHb), total hemoglobin (tHb), respiration rate, glucose, and/or otherwise. The parameters measured by the plethysmograph sensor can display on one or more monitors the foregoing parameters individually, in groups, in trends, as combinations, or as an overall wellness or other index.

Sensors available today all work well in a medical setting, and the close proximity to a processor with the medical facility ensures the system will work well. The most applicable system available today would be a watch bearing health sensor device. As an example, optical physiological sensors can be integrated into a watch configured to monitor health of a wearer. The optical physiological sensor can be configured to face tissue of the wearer when the watch is worn by the wearer and to measure physiological parameters of the wearer using information from the optical physiological sensor. The optical physiological sensor can comprise a first emitter grouping comprising a first plurality of light emitting diodes (LEDs) at a first location; a second emitter grouping comprising a second plurality of LEDs at a second location different from the first location, wherein the second emitter grouping can comprise the same number and type of LEDs as the first emitter groupings; one or more light blocks separating the first emitter grouping from the second emitter grouping; light diffusing material configured to diffuse light emitted by each of the first and second pluralities of LEDs; a plurality of detectors including four or more photodiodes; and a convex surface configured to be positioned between (i) the first and second emitter groupings and the four or more photodiodes and (ii) the tissue of the wearer, the convex surface comprising one or more surface materials.

In some configurations of this method, the patient monitoring display can be configured to display the wearer's SpO2 and pulse rate that are monitored by the sensor. In some configurations of this method, the sensor can be configured to continuously monitor the wearer's SpO2 and pulse rate. In some configurations, the display can be configured to continuously display the wearer's SpO2 and pulse rate. In some configurations of this method the watch can further comprise an ECG sensor. In some configurations of this method, the ECG sensor can comprise a reference electrode, a negative electrode, and a positive electrode.

Multiple physiological parameters, combined, provide a more powerful patient condition assessment tool than when any physiological parameter is used by itself. For example, a combination of parameters can provide greater confidence if an alarm condition is occurring. More importantly, such a combination can be used to give an early warning of a slowly deteriorating patient condition as compared to any single parameter threshold, which may not indicate such a condition for many minutes. Conditions such as hypovolemia, hypotension, and airway obstruction may develop slowly over time. A physiological parameter system that combines multiple parameters so as to provide an early warning could identify what patients may need to be moved up quickly in the queue.

### SUMMARY—PROCESSOR

The method's processor which resides in computer hardware has one or more inputs responsive to one or more physiological sensors. The processing system may also have quality indicators relating to confidence in the inputs of the sensors. A processor is adapted to combine the parameter, quality indicators and predetermined limits for the inputs and quality indicators so as to generate alarm outputs or control outputs or both.

### SUMMARY OF THE INVENTION

During the patient intake process, prior to the patient re-entering the waiting room, each patient is given a patient monitor that communicates with processor within the medical facility; one such patient monitor could be a wrist band, a band configured to secure the physiological parameter measurement sensor on a wrist of the wearer. The medical staff at Intake will set a variable threshold for key readings from each which will trigger alerts and alarms on the displace and in some case generate an audible alarm. In some implementations, the hardware processor is configured to: obtain a first plurality of signals from the physiological parameter measurement sensor when the band is secured on the wrist at a first tightness; determine a signal quality responsive to the first plurality of signals; and output an indication on the display to adjust tightness of the band with respect to the wrist from the first tightness to a second tightness based on the determined signal quality. Other patient sensors could also be used.

A display within the medical facility can show the monitoring of every patient, plus it can show alarms when a patient is in distress, or alert medical staff when a vital sign has changed.

### Additional Potential Embodiments

The one or more hardware processors are further configured to control a medical device based on the generated information from the patient monitor.

### BRIEF DESCRIPTION OF THE DRAWINGS

FIG. 1 shows one of the markets available patient monitoring devices, in this device the patient's oxygen level and heart rate would be posted. This device is for an example only of a patient monitoring device, any patient monitoring device can be used in this method.

FIG. 2 is a diagram of the information flows to the processor from the patient monitoring, to the information posted on a display and then alarms that show on the display and auditory alarms.

### DETAILED DESCRIPTION

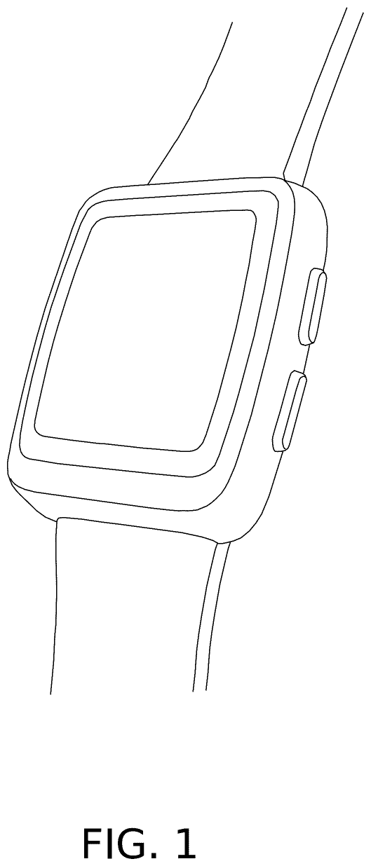

FIG. 1 shows an example of a type of patient monitoring system, but any patient monitoring system chosen by the medical facility can be used.

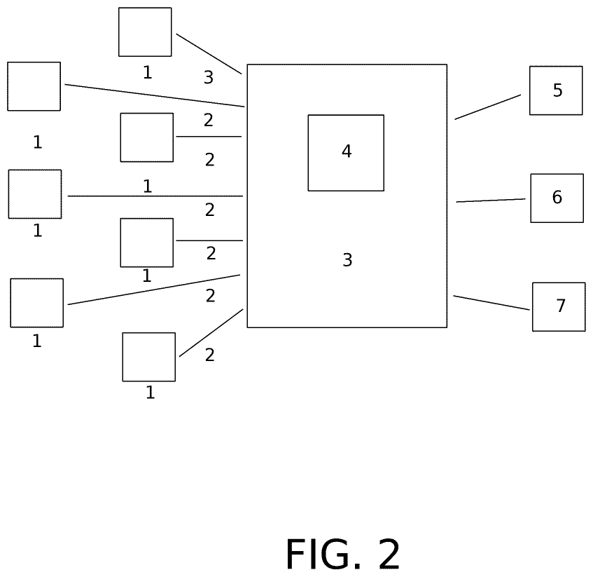

FIG. 2 shows a block diagram. The medical intake personnel enter intake data, including any thresholds for alarms, and also monitoring required on the patent monitor through a keyboard 5. Then the patients return to the waiting room where their patient monitors 1 communicate through a wireless system 2 with the hardware 3, which has the processor 4 within the hardware 3. Information regarding patients is posted on the display 6, and the display will also show alerts and alarms. The processor 3 will also activate an auditory alarm 7, when a patient exceeds a critical threshold. The processor takes input from the medical staff, but also has standard patient thresholds stored in memory so that alarms can also be initiated by standard threshold levels.

I claim

1. A method to monitor patients in a medical waiting area comprised of:
Giving to patients during intake before they wait to see a medical professional, a patent monitor, the patient monitor comprising
a physiological sensor configured to measure physiological parameters of the wearer comprising one or more of the following parameters;
SpO2 levels;
pulse rate;
respiratory rate;
ECG profile;
temperature;
In the medical office hardware with a processor with stored alert and alarm thresholds and a display that receives input from each patient monitor that is deployed;
receive at least one parameter reading from the physiological sensor worn by each patient, the parameter chosen by the medical staff at intake;
receive secondary parameters from the physiological sensor worn by each patient, the parameter chosen by the medical staff at intake;
the medical staff to generate a variable threshold, in addition to stored thresholds, for each parameter chosen for each patient, the variable threshold to set off alarms on the display;
the hardware to have an audible alert or alarm when a patient's condition deteriorates beyond the variable threshold chosen up intake by the medical staff.

2. The patient monitor of claim 1, wherein the one or more hardware processors are further configured to control a medical device based on the generated diagnosis.
